(19) United States
(12) Patent Application Publication
(10) Pub. No.: US 2025/0366709 A1
(43) Pub. Date: Dec. 4, 2025

(54) STEERABLE CATHETER DEVICE WITH VIDEO CAPABILITIES AND METHODS OF TREATMENT USING SAME

(71) Applicant: Nuvaira, Inc., Minneapolis, MN (US)

(72) Inventors: Larry Wales, Maplewood, MN (US); Ryan Kaveckis, Minneapolis, MN (US); Philip J. Johnson, Plymouth, MN (US)

(21) Appl. No.: 18/873,656

(22) PCT Filed: Jun. 12, 2023
(86) PCT No.: PCT/US2023/025087

§ 371 Date: Dec. 10, 2024

Related U.S. Application Data
(60) Provisional application No. 63/351,173, filed on Jun. 10, 2022.

(51) Int. Cl. A61B 1/267; A61B 1/005; A61B 18/00; A61B 18/14

(52) U.S. Cl. CPC A61B 1/2676; A61B 1/0052; A61B 1/0057; A61B 18/1492; A61B 2018/00023; A61B 2018/00166; A61B 2018/0022; A61B 2018/00541; A61B 2018/00577; A61B 2018/00797; A61B 2018/00821; A61B 2018/00982; A61B 2218/007

(57) ABSTRACT

A steerable sheath and catheter system includes a steerable sheath assembly with a camera or visual capabilities and a treatment catheter. The sheath assembly is optimized to be compatible with the treatment catheter and provides one or more working channels, and visualization capabilities during a procedure. The sheath assembly can also provide steering capabilities which allows the treatment catheter to gain access to a lumen of a subject, such as an airway, and be precisely positioned in the lumen for treatment thereof.

### RELATED APPLICATION

The present application claims the benefit of U.S. Provisional Application No. 63/351,173 filed Jun. 10, 2022, which is hereby incorporated herein in its entirety by reference.

### TECHNICAL FIELD

The present disclosure is directed to an apparatus having an elongate body that can be passed into an airway or lumen of a subject, allowing a user to navigate via a vision system to allow the user to pass instrumentation and devices to treatment sites.

### BACKGROUND

Physicians use tools, such as a bronchoscope or endoscope, for everyday procedures to visualize lumens or cavities and allow additional tools such as a catheter or instrument to be passed through a working channel of the scope to perform diagnostics or treatments. Today's bronchoscopes have many features, such as manipulation in multiple directions, rotational working channels, high definition (HD) quality vision systems, along with other features.

Bronchoscopes typically have working channels that have diameters from 2.8 mm to 3.2 mm. However, as medical device technology grows, there is a need for larger working channel without sacrificing features such as steerability, camera capabilities, and lighting. Working channel diameter and maintaining compatibility with the different bronchoscope manufacturers can limit the development of a technology.

Many of these features are not necessary for some procedures, so a working sheath camera sheath/steerable catheter with video capabilities can encompass the needs of specific procedures. The cost of maintaining and the robustness of a typical bronchoscope also presents challenges, which could be eliminated with a lower cost disposable type working sheath.

There remains a need for a more versatile scope system configured to be used with treatment catheters, without sacrificing the features such as visualization, flexibility, and steerability of traditional scope systems.

### SUMMARY OF THE DISCLOSURE

In examples described below, a steerable sheath and catheter system includes a steerable sheath assembly with a camera or visual capabilities, and a treatment catheter. The sheath assembly is optimized to be compatible with the treatment catheter and provides one or more working channels, and visualization capabilities during the procedure. The sheath assembly can also provide steering capabilities which allows the treatment catheter to gain access to a lumen of a subject, such as an airway, and be precisely positioned in the lumen.

In examples, the sheath assembly can include a flexible sheath that has large working channel diameter of from about 3.0 mm to about 7.0 mm, and more specifically, from about 3.9 mm to about 6 mm. The flexible sheath has at least one working channel extending through a length of the sheath. The flexible sheath can include a visualization assembly on an end of the sheath, which can include at least one camera or imaging source, and one or more sources of light, such as an LED light.

In examples, the sheath can further include grooves along its length that allow for ventilation around the sheath during a procedure, such as an intubated procedure when an airway is blocked by sheath. In examples, the sheath can optionally be used for suction of the airways when the working channel does not house the treatment catheter.

In examples, in which the treatment catheter can include a flexible shaft, and an expandable or inflatable member coupled to a distal end of the shaft, and one or more energy emitting elements coupled to the inflatable member. In examples, the camera of the sheath can be positioned on the end of the sheath such that it precisely coordinated with the length of the catheter to allow for optical coupling with the expandable member upon inflation without further manipulation of the sheath and catheter.

In another example, the sheath and catheter can be a single steerable catheter device. The working end or energy emitting, optionally expandable, end of the catheter can be incorporated into the flexible sheath itself, rather than coupled to it. This working end can be retracted into and extended out from an end of the sheath. In examples, the working end can be manipulated while extended. This configuration can optionally incorporate suction through another port or channel defined in the sheath.

In example, this configuration can include multiple cameras to visualize from the proximal and distal perspective of the working end of the catheter. The addition of a distal perspective could aid in electrode or energy emitter placement in the airway. The handle of the steerable catheter device can allow for steering the catheter, extending and retracting the working end of the catheter, and rotating the working end of the catheter. The handle can further include a locking mechanism that would lock in the deflection angle of the catheter tip to stabilize the working end of the catheter in the center of the lumen or airway. This can improve the ease with which the electrode or energy emitter is manipulated in space during the procedure.

In examples, the sheath can be specifically designed to help access difficult or tortuous anatomy, such as for example, the many branches of the airway, which is currently limited by commercially available bronchoscopes. In one example, the tortuous anatomy is the right upper lobe bronchi. Here, the airway comes off at a near 90 degree angle from the right mainstem and thus limits the ability to treat in the right upper lobe bronchi. A sheath or flexible catheter system according to examples can be developed that specifically addresses this limitation and allows access to the right upper lobe bronchus.

The above summary of the various representative examples is not intended to describe each illustrated embodiment or every implementation of the disclosure. Rather, the examples are chosen and described so that others skilled in the art can appreciate and understand the principles and practices of the invention. The figures in the detailed description that follow more particularly exemplify these embodiments.

### BRIEF DESCRIPTION OF THE DRAWINGS

The disclosure can be more completely understood in consideration of the following detailed description of various embodiments of the disclosure, in connection with the accompanying drawings, in which:

FIG. 1 is an isometric view of a camera sheath assembly according to an example of the disclosure;

FIG. 2 is an isometric view of a distal end of the camera sheath assembly of FIG. 1.

FIG. 3 is an isometric view showing the ability of the distal shaft of the camera sheath assembly to flex when activated by a flex lever on a handle of a camera sheath assembly, according to an example of the disclosure.

FIG. 4 is an isometric view showing a balloon catheter assembly deployed and attached to the camera sheath assembly of FIG. 1.

### DETAILED DESCRIPTION

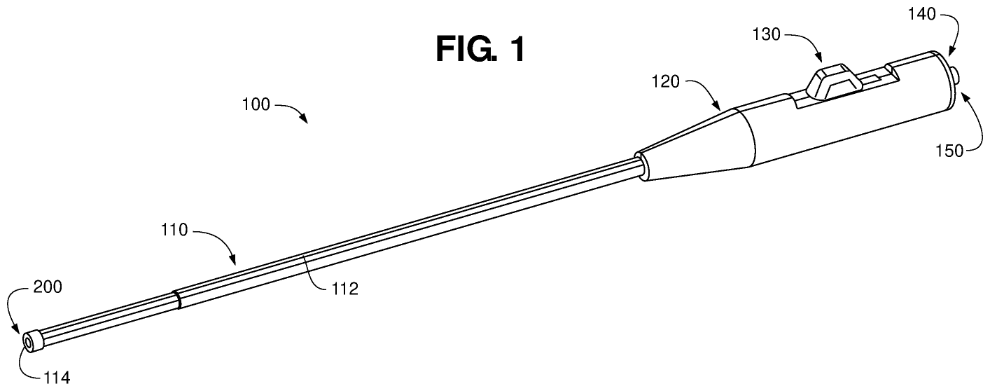

Referring to FIGS. 1-3, a camera sheath assembly 100 includes an elongate working shaft 110, a handle 120 coupled to a proximal end of shaft 110, flex lever 130 for flexing a flexible distal end of shaft 110 at a range of angles from shaft 110, port 140 for coupling a treatment catheter thereto, camera and light connection port 150 for coupling an imaging or visualization source with display thereto, and a distal assembly 200 including one or more cameras, channels, and/or light sources.

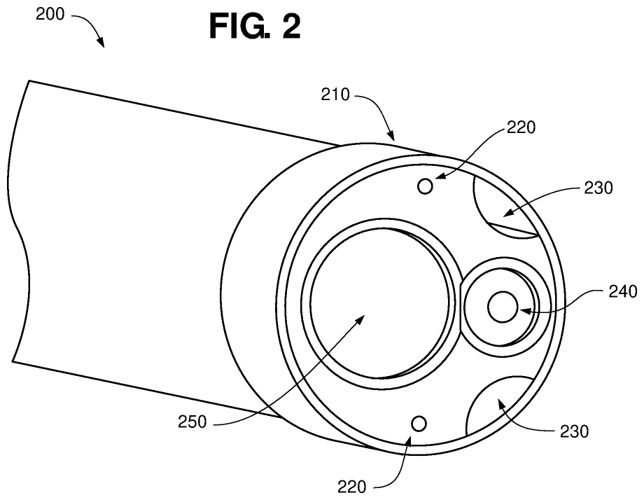

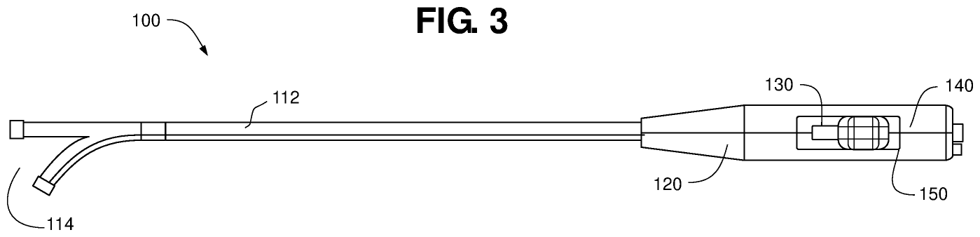

In examples, the working shaft 110 is a coextruded tube with structure defining multiple lumens extending along its length from the proximal end to the distal end, which will be described in more detail with respect to FIG. 2. In one example, the coextruded tube has a stiffer proximal section 112, and a softer flexible distal section 114 such that the softer flexible section can bend at an angle with respect to stiffer proximal section 112 (see. for example. FIG. 3). The stiffer proximal section 112 gives the shaft push ability, while the soft distal section 114 can flex to help steer the direction of the catheter (not shown). In examples. the working shaft 110 can he comprise braided Nitinol® or stainless-steel wires to help with structure and performance such as torque and push ability. The braid angle may be tighter or higher in the stiffer proximal section 112 than the distal section 114 to provide its stiffness.

The handle 120 gives the user a means to hold onto the shaft 110, while providing enclosures for pull wires, cam actuators, electrical wires, and other components of assembly 100. The flex lever 130 is attached to a cam mechanism (not shown) that either pushes or pulls on wires to flex the distal section 114 of the shaft 110 with respect to the proximal section 112, when the lever 130 is actuated. The flex lever 130 depicted can be thumb or finger activated lever. Alternatively, it could be designed as a trigger type lever activated by a finger pull.

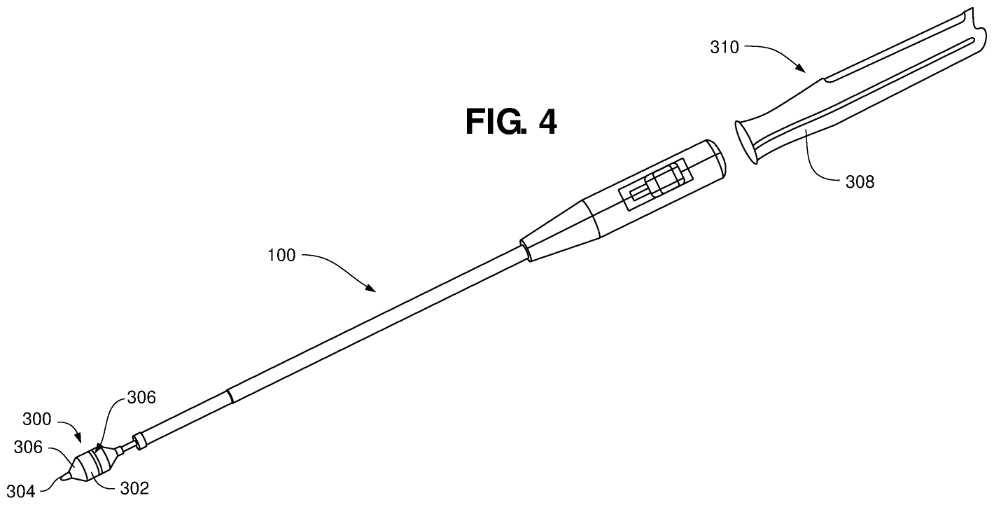

The port 140 allows access to the working channel of the shaft 110 and allows a handle of a treatment catheter to directly attach thereto, similar to attachment of the handle of a treatment catheter and a port of a bronchoscope. A suitable treatment catheter and handle assembly is described, for example, in U.S. Pat. App. Pub. No. 2016/0310210, entitled “Catheter and Handle Assembly, Systems, and Methods”, incorporated herein by reference in its entirety. As depicted in FIG. 4, the handle assembly 310 is removable coupled to port 140 of handle 120 such that an elongate catheter shaft (not shown) of a treatment catheter assembly 300 is introduced and extends into and through the working channel of shaft 110 such that a treatment device, depicted as a balloon catheter, extends from the distal end of shaft 110.

The handle assembly 310 can be coupled to handle 120 in any of a variety of ways such as be snap lock or friction fit, threaded engagement of handle 120 and assembly 310, or other mechanisms as described. for example. in U.S. Pat. App. Pub. No. 2016/0310210. In examples. a treatment catheter assembly 300 is coupled to a handle assembly 310. In an example, the catheter assembly 300 can comprise a targeted lung denervation RF, microwave, or ultrasound catheter, and generally includes an elongate shaft 302, and an ablation assembly 304 coupled to a distal portion of the shaft 302, the ablation assembly 304 including an expandable member, such as a balloon or basket, and one or more electrodes or energy emitters 306 coupled to the expandable member 304. The catheter assembly 300 also includes a cooling circuit including a coolant inflow and outflow lumen within the elongate shaft, and a coolant inlet path and return path (e.g. cooling conduit(s)) to circulate coolant to the expandable member 304 and to the energy emitter 306, one or more power wires for supplying power to the energy emitter 306, optional thermocouple(s) and associated wires for measuring and sensing temperature at locations proximal to the electrode, optional cooling circuit pressure sensors and associated wired for measuring and sensing pressure within the cooling circuit, and/or optional pressure relief valves.

In examples, the handle assembly 310 is coupled to a proximal portion of the shaft 302. The handle assembly 310 can include a housing 308 fixedly coupled to the proximal end of the shaft 302, and a spindle tube or handle frame (not shown) coupled to the housing such that the spindle tube is rotatably and axially shiftable with respect to the housing and the catheter assembly 300. The handle assembly 310 can further include an umbilical cable with strain relief for coupling the handle assembly 310, and ultimately the catheter assembly 300, to a system console including a heat exchanger, coolant pump, energy generator (such as an RF, microwave, or ultrasound generator), and a system controller. The umbilical cable can aid in coupling, for example, inlet and return fluid tubes (coolant) from the system console for fluidly coupling the catheter assembly 300 to the heat exchanger and pump of the console. an electrical cable/connector to electrically connect the electrode 306 of the catheter assembly 300 to an energy source. thermocouple wires to monitor temperature of the surface tissue of the treatment site, the electrode, or both, and/or pressure sensors to monitor the high pressure coolant inlet flow and the low pressure return flow.

Referring back to FIGS. 1 and 2, the camera and light connection 150 allow a visualization system 200, such as a camera and light positioned on the distal end of shaft 110, to connect to a processor board to create a display for displaying real time navigation of a lumen in which the treatment catheter is deployed.

More specifically, FIG. 2 is an isometric view of the distal end of the camera sheath that can include the following components of the visualization system 200: distal cap 210, one or more pull wire 220, camera and light assembly 240, and working channel 250. In this non-limiting example, the multi-lumen extrusion 200 has four lumens and two air channels 230; however more or less can be contemplated. The air channels 230 are used to distribute air to the lungs when the sheath 100 is positioned into the airway.

The distal cap 210 can comprise a stainless-steel cap that allows attachment of the pull wires 220. The pull wires 220 are fixedly attached to the end cap 210, via a process such as but not limited to, laser welding, or brazing. The geometry of the end cap 210 is such that it captures the surface of the distal end of the extrusion allowing it to flex when tension is applied to the pull wire 220. Two pull wires are shown in the image; however, a polarity of pull wires could be used to flex in multiple directions. The camera and light assembly 240 is positioned within one of the lumens in the extrusion. The camera and light assembly 240 is used to visualize into a cavity allowing the user to navigate the distal end of the sheath 100 to a treatment location. The camera and light assembly 240 can be fixedly attached within the lumen creating a disposable mechanism. An alternative option is to use a scope such as, but not limited to a fiber scope, that can slide into the lumen and be removed from the lumen. This option allows the user to reuse the scope and dispose of the sheath only. The working channel 250 allows passage of instruments and devices into the treatment site, such as the shaft 302 and ablation assembly 403 of catheter assembly 300.

Referring now to FIG. 3, shaft 110 includes a flexible portion 112 that has the ability to flex the distal end by manipulating the flex lever 130 to pull on a wire (220, FIG. 2) that is attached to the distal cap (210) and is constrained within the lumens of the extruded shaft (110).

The systems according to embodiments can be used to maneuver ablation assemblies comprising expandable members and one or more energy emitters or electrodes as described above, and/or can be designed or configured for axially, rotational, and/or other manipulation of any of a variety of treatment assemblies including needle and/or needleless injection or drug delivery systems such as, for example, for the injection or delivery of neurotoxins, sclerosing agents, any of a variety of agents for the treatment of pulmonary disorders, without the need for an introduction device, such as a bronchoscope. For example, the handle and catheter system can be configured to axially (advancing and retracting) and/or rotationally steer or manipulate one or more needles or ports within and around the airway. while visualizing the system from one or more perspectives. Various non-limiting examples of assemblies are described in one or more of the patents and applications listed below, all of which are incorporated by reference in their entireties herein:
U.S. Pat. No. 8,088,127 entitled “Systems. Assemblies, and Method for Treating a Bronchial Tree;”
U.S. Patent Application Publication No. 2011/0152955 entitled “Delivery Devices with Coolable Energy Emitting Assemblies;”
U.S. Patent Application Publication No. 2012/0310233 entitled “Systems, Apparatus, and Methods for Treating Tissue and Controlling Stenosis;”
U.S. Patent Application Publication No. 2011/0118725 entitled Non-invasive and Minimally Invasive Denervation Methods and Systems for Performing the Same;”
U.S. Patent Application Publication No. 2012/0302909 entitled “Methods and Systems for Screening Subjects;”
U.S. Patent Application Publication No. 2011/0301587 entitled “System and Method for Pulmonary Treatment;”
U.S. Pat. No. 8,172,827 entitled “Apparatus for Treating Asthma Using a Neurotoxin;”
U.S. Patent Application Publication No. entitled “Method and Apparatus for Controlling
U.S. Pat. No. 8,483,831 entitled “System and Method for Bronchial Dilation;”
PCT Application Publication No. WO 2013/052501 entitled “Apparatuses and Methods for Injuring Nerve Tissue;”
U.S. Patent Application Publication No. 2013/0310822 entitled “Compact Delivery Pulmonary Treatment System and Method for Improving Pulmonary Function;”
U.S. Provisional Patent Application No. 61/746,460 entitled “Methods for Improving Drug Efficacy;”
U.S. Provisional Patent Application No. 61/779,371 entitled “Fluid Delivery System and Method for Treatment;”
U.S. Provisional Patent Application No. 61/876,925 entitled “Systems, Devices, and Methods for Treating a Pulmonary Disease with Ultrasound Energy;”
U.S. Provisional Patent Application No. 61/847,477 entitled “Methods for Protecting the Esophagus During Pulmonary Treatment Procedures: ” and
U.S. Provisional Patent Application Nos. 61/799,742 and 61/870,373, both entitled “Systems, Devices, and Methods for Treating a Pulmonary Disorder with an Agent.”

It should also be appreciated that the exemplary embodiment or exemplary embodiments are only examples, and are not intended to limit the scope, applicability, or configuration of the subject matter hereof in any way. Rather, the foregoing detailed description will provide those skilled in the art with an enabling disclosure for implementing the exemplary embodiment or exemplary embodiments. It should be understood that various changes can be made in the function and arrangement of elements without departing from the scope of the subject matter hereof as set forth in the appended claims and the legal equivalents thereof.

The embodiments above are intended to be illustrative and not limiting. Additional embodiments are within the claims. Although the present subject matter has been described with reference to particular embodiments, workers skilled in the art will recognize that changes may be made in form and detail without departing from the spirit and scope of the subject matter hereof.

Various modifications to the subject matter hereof may be apparent to one of skill in the art upon reading this disclosure. For example, persons of ordinary skill in the relevant art will recognize that the various features described for the different embodiments of the subject matter can be suitably combined, un-combined, and re-combined with other features. alone. or in different combinations, within the spirit of the subject matter hereof. Likewise, the various features described above should all be regarded as example embodiments, rather than limitations to the scope or spirit of the subject matter hereof. Therefore, the above is not contemplated to limit the scope of the present subject matter hereof.

For purposes of interpreting the claims for the present subject matter, it is expressly intended that the provisions of Section 112, sixth paragraph of 35 U.S.C. are not to be invoked unless the specific terms “means for” or “stop for” are recited in a claim.

1. A camera sheath assembly comprising:
an elongate shaft having a proximal end and a distal end, and having structure defining a working channel extending from the proximal end to the distal end;
a handle coupled to the proximal end of the shaft, the handle being configured to operably couple to a treatment catheter;
a visualization assembly coupled to the second end of the shaft, the visualization assembly including at least one camera and at least one source of light; and
at least one pull wire configured to direct the distal end in one or a plurality of directions.

2. The camera sheath assembly of claim 1, further comprising:
a port configured to be coupled to a handle of the treatment catheter to allow access to the working channel within the shaft, which a shaft and an ablation assembly of the treatment catheter are configured to extend within the working channel with the port is coupled to the handle.

3. The camera sheath assembly of claim 1, wherein the handle of the camera sheath assembly comprises:
an actuation lever to assist in the maneuvering of the least one pull wire residing within the working channel of the shaft to initiate a distal portion of the shaft to flex relative to a proximal portion of the shaft.

4. The camera sheath assembly of claim 3, wherein the actuation lever comprises a thumb or finger activation lever, or a trigger activated by finger pull.

5. The camera sheath assembly of claim 3, wherein the distal portion of the shaft has a flexibility greater than the proximal portion of the shaft.

6. The camera sheath assembly of claim 5, wherein the shaft is formed of a braided jacket, and a braid angle of the distal portion is less than a braid angle of the proximal portion.

7. The camera sheath assembly of claim 3, wherein the proximal portion has a length greater than a length of the distal portion.

8. The camera sheath assembly of claim 1, wherein the shaft is a coextruded tube comprising multiple lumens.

9. The camera sheath assembly of claim 1, wherein the shaft further comprises:
a plurality of air channels for distribution of air into the treatment locations; and
a distal cap which allows for assembly and attachment of a plurality of pull wires.

10. The camera sheath assembly of claim 9, wherein the pull wires are attached to the distal cap via laser welding or brazing.

11. The camera sheath assembly of claim 1, wherein the handle further comprises a camera/light port to signal data received from the visualization assembly to a receiving processor board to generate display imaging data for visualization and navigation to a treatment location.

12. The camera sheath assembly of claim 1, wherein the working channel has a diameter of from about 3.9 mm to about 6 mm.

13. The camera sheath assembly of claim 1, wherein the shaft further comprises grooves defined on an outer surface thereof, the grooves being configured to allow for ventilation around the sheath during an intubated procedure.

14. The camera sheath assembly of claim 1, wherein the camera sheath assembly is configured to suction a lumen of a subject when the working channel does not house the treatment catheter.

15. The camera sheath assembly of claim 1, wherein the camera sheath and the catheter device can operate as a single steerable catheter device by which a working end of the treatment catheter is incorporated into the shaft of the assembly.

16. The camera sheath assembly of claim 15, wherein the incorporation of the shaft of the camera sheath assembly and the treatment catheter is configured to have a plurality of cameras for visualizing the proximal and distal perspective of the working end of the treatment catheter.

17. A method of accessing treatment sites, comprising:
coupling the treatment catheter to the camera sheath assembly of claim 1; and
steering the treatment catheter to a treatment site via the handle, wherein a shaft of the treatment catheter is contained within the working channel of the shaft, and wherein the length of the catheter providing optimal coupling for visualization and steering ability during a procedure through which the catheter gains access into the airway with precise positioning.

18. The method of claim 17, wherein a working end of the shaft of the treatment catheter comprises an expandable member having at least one energy emitter positioned thereon, the method further comprising:
advancing the expandable member in a retracted state through the working channel and the distal end of the shaft; and
expanding the expandable member to an expanded state,
wherein the expandable member and a position thereof is viewable on a display screen operably connected to the visualization assembly.

19. The method of claim 18, the method further comprising:
delivering energy to from the at least one energy emitter of the expandable member to a first treatment site to treat a condition of the airway.

20. The method of claim 19, the method further comprising:
moving the at least one energy emitter to a second treatment site while displaying the same on the display screen; and
delivering energy to from the at least one energy emitter of the expandable member to a first treatment site to treat a condition of the airway.

21. The method of claim 19, the method further comprising:
providing air to the airway via airway channels formed in the shaft while delivering energy to the treatment site.
